(19) United States
(12) Patent Application Publication
(10) Pub. No.: US 2026/0335062 A1
(43) Pub. Date: Oct. 29, 2026

(54) HEART VALVE DELIVERY SYSTEMS AND PACKAGING SOLUTIONS

(71) Applicant: Edwards Lifesciences Corporation, Irvine, CA (US)

(72) Inventor: Eric Robert Dixon, Villa Park, CA (US)

(21) Appl. No.: 19/699,735

(22) Filed: Jun. 5, 2026

Related U.S. Application Data
(63) Continuation of application No. PCT/US2024/058710, filed on Dec. 5, 2024.
(60) Provisional application No. 63/607,662, filed on Dec. 8, 2023.

(51) Int. Cl. A61F 2/00; A61F 2/24

(52) U.S. Cl. CPC A61F 2/0095; A61F 2/2427

(57) ABSTRACT

Systems apparatuses and assemblies for tilting and flushing delivery systems. The assembly can include an implant, a delivery system, and packaging. The delivery system can include a catheter, a control handle, and a distal part coupled to the implant. The control handle can extend from a proximal end of the catheter. The packaging can include a first portion, a second portion and a lateral connecting the first portion and the second portion. The first portion can contain the implant. The second portion can contain the handle. The lateral line can enable the first portion to be rotated relative to the second portion. Rotating the first portion can form the packaging into a tilt stand in which the handle is tilted to facilitate flushing of the delivery system and the implant via the fluid port.

### CROSS-REFERENCE TO PRIORITY APPLICATIONS

The present application is a continuation of International Application No. PCT/US2024/058710, filed Dec. 5, 2024, which designates the United States and was published in English by the International Bureau on Jun. 12, 2025, as WO2025/122773, which claims priority to U.S. Provisional Application No. 63/607,662 filed Dec. 8, 2023. The entire content of each of the above is hereby incorporated herein by reference.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Prostheses can be used to correct problems associated with impaired heart valves. For example, mechanical and tissue-based heart valve prostheses can be used to replace impaired native heart valves. More recently, substantial effort has been dedicated to developing replacement heart valves that can be delivered with less trauma to the patient than through open heart surgery. Replacement valves can be designed for percutaneous delivery in so-called transcatheter procedures.

Development of transcatheter heart valves and their delivery systems has proven to be challenging. Further, delivery systems should be free of air bubbles to help prevent formation of potentially dangerous air embolisms in the bloodstream. Pushing liquid through lumens of a delivery system to move bubbles out of the system can be difficult, especially if very small lumens are involved. Loading a device in a delivery system could potentially introduce bubbles if the user is not careful enough as well.

In light of the above, a need exists for systems, devices, and methods for preparing delivery systems, including for optimal removal of potential air bubbles.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In some implementations, systems, devices, apparatuses, assemblies, methods, etc. for flushing a delivery system (e.g. a transcatheter delivery system, a transcatheter heart valve delivery system, a device delivery system, a treatment device delivery system, a repair device delivery system, an implant delivery system, etc.) are disclosed. In some implementations, a device (e.g., a treatment device, an implant, a replacement device, a heart valve, a prosthetic heart valve, a repair device, etc.) can be coupled to the delivery system during flushing.

In some implementations, a flushing assembly (e.g., for use in or useable for flushing a delivery system) includes a lower hub. In some implementations, the lower hub has a fluid port.

In some implementations, the lower hub is connected to an elongated tube extending upward to an immersion tank (e.g., a liquid tank, a liquid trough, a trough, a liquid compartment, a compartment, etc.). In some implementations, an aligned through bore extends from the hub to the immersion tank. In some implementations, the through bore permits introduction of a catheter (e.g., a distal catheter, etc.) of the delivery system such that a distal end of the catheter can be introduced into the immersion tank.

In some implementations, the device (e.g., treatment device, implant, replacement device, heart valve, prosthetic heart valve, repair device, etc.) can be expanded and immersed in fluid in the immersion tank while fluid or liquid is introduced to a control handle to flush the delivery system (e.g., in an upward direction, etc.).

In some implementations, the flushing assembly can be packaged with the delivery system or provided separately.

In some implementations, various packaging solutions can incorporate a flushing assembly or portions thereof that can be folded to provide a tilt stand to facilitate a flushing operation.

In some implementations, an assembly (e.g., a first assembly of a system, etc., which can optionally be in addition to one or more other assemblies of the system) for flushing a delivery system comprises a delivery system (e.g. a transcatheter delivery system, a transcatheter heart valve delivery system, a device delivery system, a treatment device delivery system, a repair device delivery system, an implant delivery system, etc.) having a proximal control handle and a catheter that extends from (e.g., relative to) a distal end of the control handle.

In some implementations, the delivery system has movable elements to which an expandable/collapsible device (e.g., treatment device, implant, replacement device, heart valve, prosthetic heart valve, repair device, etc.) can be coupled. In some implementations, the movable elements can be configured to retract and collapse the device relative to a distal end of the catheter. In some implementations, the movable elements can be configured to expel and expand the device relative to a distal end of the catheter. In some implementations, the movable elements can be configured to alternately retract and collapse or expel and expand the device relative to a distal end of the catheter.

In some implementations, a flushing implement comprises an elongated tube terminating at a lower end in a hub. In some implementations, the hub and elongated tube comprise aligned through bores sized to slidingly receive the catheter.

In some implementations, the assembly (e.g., first assembly, etc.) and/or the flushing implement includes an immersion tank (e.g., a liquid tank, a liquid trough, a trough, a liquid compartment, a compartment, etc.) located or positionable at an upper end of the tube. In some implementations, the system or assembly is configured such that a catheter can be inserted from below into the hub and advanced upward until a distal end thereof is within the immersion tank.

In some implementations, the device can be expanded and immersed in fluid/liquid held by gravity in the immersion tank such that fluid/liquid introduced to the control handle flushes the catheter from the control handle upward.

In some implementations, in the first assembly, the hub can have a fluid port in fluid communication with a concentric space between the tube and the catheter. In some implementations, the assembly can have a fluid coupling and line with a fluid release valve therein. In some implementations, the fluid coupling and/or line are attached to the fluid port to permit selective draining of fluid from the immersion tank and through the concentric space.

In some implementations, the hub can have a plurality of elastomeric valves therein. In some implementations, the flushing implement can have a loader that removably clips onto the hub. In some implementations, the flushing implement can have a forward nose piece sized to pass through the elastomeric valves in the hub to facilitate insertion of the catheter into the tube.

In some implementations, the assembly (e.g., first assembly, etc.) can further include packaging with recesses sized and shaped to cradle the delivery system when inserted into the flushing implement. In some implementations, the assembly (e.g., the packaging thereof) can include an enlarged cavity sized to cradle (and/or form) the immersion tank.

In some implementations, the assembly can include the device (e.g., treatment device, implant, heart valve, repair device, etc.). In some implementations, the device is coupled to the delivery system and stored within the packaging.

In some implementations, the assembly can include an introducer with a proximal hub. In some implementations, the assembly (e.g., the introducer thereof, etc.) can include a distal access sheath. In some implementations, two or more of the introducer, proximal hub, and/or distal access sheath can be assembled/arranged/positioned concentrically over the catheter.

In some implementations, the introducer is sized to fit through the flushing implement. In some implementations, the hub also has a fluid port in fluid communication with internal passages within the access sheath such that fluid introduced to the introducer fill port flushes the access sheath upward.

In some implementations, an assembly (e.g., a second assembly of a system, etc., which can optionally be in addition to a first assembly and/or one or more additional assemblies of the system) for flushing a delivery system comprises a delivery system (e.g. a transcatheter delivery system, a transcatheter heart valve delivery system, a device delivery system, a treatment device delivery system, a repair device delivery system, an implant delivery system, etc.) with a proximal control handle. In some implementations, the delivery system comprises a catheter that extends from or relative to a distal end of the control handle.

In some implementations, the delivery system has movable elements to which an expandable/collapsible device (e.g., treatment device, implant, replacement device, heart valve, prosthetic heart valve, repair device, etc.) can be coupled. In some implementations, the movable elements can be configured to retract and collapse the device relative to a distal end of the catheter. In some implementations, the movable elements can be configured to expel and expand the device relative to a distal end of the catheter. In some implementations, the movable elements can be configured to alternately retract and collapse or expel and expand the device relative to a distal end of the catheter.

In some implementations, a flushing implement comprises an elongated tube terminating at a lower end in a hub. In some implementations, the hub and tube have aligned through bores sized to slidingly receive the catheter.

In some implementations, the flushing implement includes an immersion tank (e.g., a liquid tank, a liquid trough, a trough, a liquid compartment, a compartment, etc.) at an upper end of the tube with a cover. In some implementations, the immersion tank or cover can have a fluid inlet to which a supply of saline is attached or is attachable.

In some implementations, the assembly is configured such that the catheter can be inserted from below into the hub and advanced upward until a distal end thereof is within the immersion tank. In some implementations, the device can be expanded and immersed in fluid introduced through the inlet into the immersion tank. In some implementations, this can be done while fluid/liquid is pulled proximally by suction from the control handle to flush the catheter downward.

In some implementations, the assembly can include an introducer with a proximal hub. In some implementations, the assembly and/or introducer can include a distal access sheath. In some implementations, one or more of the introducer, hub, and/or distal access sheath can be assembled/arranged/positioned concentrically over the catheter.

In some implementations, the introducer is sized to fit through the flushing implement. In some implementations, the hub has a fluid port in fluid communication with internal passages within the access sheath such that fluid pulled proximally by suction into the introducer fill port flushes the access sheath downward.

In some implementations, an assembly (e.g., a third assembly of a system, etc., which can optionally be in addition to a first assembly and/or a second assembly of the system) for flushing a delivery system comprises a delivery system (e.g. a transcatheter delivery system, a transcatheter heart valve delivery system, a device delivery system, a treatment device delivery system, a repair device delivery system, an implant delivery system, etc.) with a proximal control handle. In some implementations, a catheter extends from or relative to a distal end of the control handle. In some implementations, the control handle has a fluid port in communication with an inner lumen of the catheter.

In some implementations, the delivery system has movable elements therein to which an expandable/collapsible device (e.g., treatment device, implant, replacement device, heart valve, prosthetic heart valve, repair device, etc.) is coupled. In some implementations, the movable elements can be configured to retract and collapse the device relative to a distal end of the catheter. In some implementations, the movable elements can be configured to expel and expand the device relative to a distal end of the catheter.

In some implementations, the assembly comprises packaging that has recesses sized and shaped to cradle the delivery system and/or device. In some implementations, the packaging can be cut or scored along a lateral line that enables a distal portion of the packaging to be bent downward relative to a proximal portion. In some implementations, the distal portion can be bent relative to the proximal portion to form a tilt stand while the delivery system and/or device remain held within the recesses. In some implementations, this can be configured such that the device (e.g., treatment device, implant, heart valve, etc.) is elevated above the control handle. In some implementations, the device can be elevated above the control handle such that fluid introduced though the control handle fluid port and upward through the catheter flushes the catheter and/or device.

In some implementations, in the assembly (e.g., third assembly, etc.), the device coupled to the delivery system can be stored within the packaging. In some implementations, the assembly can be configured such that the device can remain positioned within a proximal portion of the packaging during flushing such that fluid used to flush the catheter and the device pools in the proximal end.

In some implementations, the proximal portion of the packaging can have a pour spout formed therein at one proximal corner to facilitate pouring the fluid after flushing. In some implementations, the assembly can include a drip tray held within one of the recesses of the packaging for use in catching fluid that is used to flush the catheter and/or device. In some implementations, a drip tray can be formed as an integral part of the packaging for use in catching fluid that is used to flush the catheter and/or the device. In some implementations, the drip tray can be hinged to the distal portion of the packaging.

In some implementations, the assembly (e.g., third assembly, etc.) can include an introducer with a proximal hub. In some implementations, the introducer can include a distal access sheath. In some implementations, one or more of the introducer, hub, and/or distal access sheath can be assembled/arranged/positioned concentrically over the catheter.

In some implementations, the hub has a fluid port in fluid communication with internal passages within the access sheath such that fluid introduced into the introducer fill port flushes the access sheath upward.

In some implementations, an assembly (e.g., an assembly of a system, etc., which can be one of multiple assemblies of the system or a stand-alone assembly) for flushing a delivery system comprises a delivery system (e.g. a transcatheter delivery system, a transcatheter heart valve delivery system, a device delivery system, a treatment device delivery system, a repair device delivery system, an implant delivery system, etc.) having a proximal control handle. In some implementations, a catheter extends from or relative to a distal end of the control handle. In some implementations, the control handle has a fluid port in communication with an inner lumen of the catheter.

In some implementations, the delivery system has movable elements to which an expandable/collapsible device (e.g., treatment device, implant, heart valve, repair device, etc.) is coupled. In some implementations, the movable elements can be configured to retract and collapse the device relative to a distal end of the catheter. In some implementations, the movable elements can be configured to expel and expand the device relative to a distal end of the catheter. In some implementations, the movable elements can be configured to alternately retract and collapse or expel and expand the device relative to a distal end of the catheter.

In some implementations, the assembly (and/or a system comprising the assembly) comprises packaging having recesses sized and shaped to cradle the delivery system and/or the device.

In some implementations, the device can be stored within the packaging while coupled to the delivery system. In some implementations, the delivery system and device can both be stored within the packaging while coupled together. In some implementations, the delivery system can be stored within the packaging while not coupled to the device.

In some implementations, the packaging has a distal portion that is hinged to a proximal portion. In some implementations, the distal portion is configured to be rotated relative to the proximal portion to form a tilt stand while the delivery system and device remain held within the recesses. In some implementations, the tilt stand can be configured to have the device elevated above the control handle.

In some implementations, the distal portion and proximal portion have one or more mating structures that enable them to be snapped together when the distal portion is rotated to form the tilt stand. In some implementations, the tilt stand can be configured such that fluid introduced though the control handle fluid port and upward through the catheter flushes the catheter and/or the device.

In some implementations, the packaging that is cut or scored along a lateral line that enables the proximal portion of the packaging to be bent downward relative to the distal portion to form the tilt stand while the delivery system and the device remain held within the recesses. In some implementations, this can be done with the device elevated below the control handle. In some implementations, the assembly and/or tilt stand is configured such that fluid introduced into the distal portion immerses the device. In some implementations, the assembly and/or tilt stand is configured such that fluid pulled by suction from the control handle fluid port and upward through the catheter flushes the catheter and the device. In some implementations, the assembly and/or tilt stand is configured such that fluid introduced though the control handle fluid port and downward through the catheter flushes the catheter and the device.

In some implementations, the assembly and/or tilt stand is configured such that fluid introduced into the distal portion immerses the device, and fluid pulled by suction from the control handle fluid port and upward through the catheter flushes the catheter and the device, and/or fluid introduced though the control handle fluid port and downward through the catheter flushes the catheter and the device.

In some implementations, the packaging is cut or scored along a first lateral line and a second lateral line. In some implementations, the first lateral line is located across the control handle of the delivery system. In some implementations, the second lateral line is just proximal to the device.

In some implementations, the first lateral line and the second lateral line enable both proximal and distal portions of the packaging to be bent downward relative to a middle portion to form a tilt stand(s). In some implementations, this can be done while the delivery system and the device remain held within the recesses with the device below the control handle. In some implementations, the assembly and/or tilt stand(s) is configured such that fluid introduced though the control handle fluid port and downward through the catheter flushes the catheter and the device. In some implementations, the assembly and/or tilt stand(s) is configured such that the device is immersed in fluid in one of the recesses. In some implementations, the assembly and/or tilt stand(s) is configured such that fluid pulled by suction from the control handle fluid port and upward through the catheter flushes the catheter and the device.

In some implementations, the packaging includes a drip tray held within one of the recesses of the packaging for use in catching fluid that is used to flush the catheter and/or the device. In some implementations, the drip tray defines a sealed aperture through which the catheter can extend in a sealed manner. In some implementations, the sealed aperture maintains a seal with the delivery system and the device remaining within the packaging. In some implementations, the sealed aperture maintains a seal with the delivery system and the device removed therefrom. In some implementations, the assembly and/or drip tray are configured such that fluid introduced though the control handle fluid port and through the catheter and into the drip tray flushes the catheter and the device.

In some implementations, the packaging forms a drip tray with one of the recesses located below the device for use in catching fluid that is used to flush the catheter and/or the device. In some implementations, the assembly and/or drip tray are configured such that fluid introduced into the drip tray with the catheter bent immerses the device in the fluid. In some implementations, the assembly and/or drip tray are configured such that fluid in the drip tray pulled by suction from the control handle fluid port and through the catheter can flush the catheter and the device. In some implementations, the assembly and/or drip tray are configured such that fluid introduced though the control handle fluid port and through the catheter into the drip tray can flush the catheter and the device. In some implementations, the assembly and/or drip tray are configured such that fluid introduced into the drip tray with the catheter bent immerses the device in the fluid, and fluid in the drip tray pulled by suction from the control handle fluid port and through the catheter flushes the catheter and the device, or fluid introduced though the control handle fluid port and through the catheter into the drip tray flushes the catheter and the device.

In some implementations, the assembly can include an introducer having a proximal hub. In some implementations, the introducer can include a distal access sheath. In some implementations, one or more of the introducer, hub, and/or distal access sheath can be assembled concentrically over the catheter.

In some implementations, the hub has a fluid port in fluid communication with internal passages within the access sheath such that fluid introduced into the introducer fill port flushes the access sheath upward.

In some implementations, the hub has a fluid port in fluid communication with internal passages within the access sheath such that fluid introduced into the introducer fill port flushes the access sheath downward.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the invention will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1 is a schematic representation of a transcatheter procedure showing advancement of a catheter or sheath through a femoral vein in the leg up to the vicinity of the mitral valve within the heart, and FIG. 1A is an enlargement of the heart showing the distal end of the catheter or sheath in the vicinity of the tricuspid valve;

FIGS. 2A and 2B are cross-sections of a heart showing steps in a mitral valve replacement procedure utilizing a valve delivery system;

FIG. 3 is an elevational view of an implementation of a device and delivery system integrated with an introducer sheath;

FIG. 4 is an elevational view of an implementation of an integrated device and delivery system where the device is tethered to a component of the delivery system, and FIG. 4A is an enlargement of the distal end of the delivery system showing the device as a heart valve;

FIG. 5 is an elevational view of a transcatheter delivery system by itself;

FIG. 6 is an elevational view of an introducer sheath separate from the delivery system of FIG. 5;

FIGS. 7A and 7B illustrate two steps in a process used to de-air a transcatheter heart valve delivery system;

FIG. 8A is an exploded view showing a delivery system next to a de-airing or flushing implement, and FIG. 8B is an assembled view of the two components;

FIG. 9 is an enlargement of the flushing implement assembled with a delivery system and showing the device expanded from the delivery system within an immersion tank;

FIG. 9A is an exploded view of an example flushing implement that uses a loader, shown in FIG. 9B. FIG. 9C shows the flushing implement assembled with the delivery system and showing a device expanded within an immersion tank;

FIGS. 10A-10E are enlargements showing steps in flushing or de-airing of a device within the immersion tank and removal of the delivery system from the immersion tank;

FIG. 11A is a schematic view of an example flushing assembly wherein saline fills an immersion tank through suction and gravity, and FIG. 11B illustrates the flushing assembly in use;

FIG. 12A is an exploded plan view of a delivery system assembled with the flushing implement and packaging therefor;

FIG. 12B shows the delivery system and flushing implement of FIG. 12A fully packaged;

FIG. 13 is a plan view of a delivery system in an example package of the present application;

FIG. 14 is a side view of the delivery system and example package of FIG. 13 showing a portion of the package folded away from the device to provide a tilt stand therefor;

FIGS. 14A and 14B are variations of the delivery system and package of FIG. 13;

FIG. 15 is a side view of a delivery system and an example package with a portion folded to provide a tilt stand therefor, wherein the delivery system and device can be flushed or de-aired while still within the package;

FIG. 16 is a perspective view of an exemplary drip tray sized to fit within a delivery system package;

FIG. 17 illustrates an example delivery system package with a pour spout on one corner, and FIG. 17A shows an enlargement of the pour spout;

FIG. 18A is a side view schematically illustrating an example delivery system package having hinged components, and FIG. 18B is a side view after the hinged components are folded outward to create both a tilt stand and a built-in drip tray;

FIG. 19A is a side view schematically illustrating a further modified delivery system package having hinged components, and FIG. 19B is a side view after the hinged components are folded outward to create both a tilt stand and a built-in drip tray;

FIG. 20 is a side view schematically showing an example delivery system package having a hinged tilt stand incorporated therein;

FIG. 21 shows the delivery system package of FIG. 20 after the tilt stand is folded out, and FIG. 21A is a sectional view showing an interference fit between portion of the folded tilt stand and an underside of the packaging;

FIG. 22 is a plan view of an example delivery system package;

FIG. 23 is a side elevational view showing a portion of the package folded down to create a tilt stand and showing how a distal end of the package forms a reservoir to facilitate aspiration of fluid and bubbles from the device and through the delivery system;

FIG. 24 is a plan view of another modified delivery system package;

FIG. 25 is a side elevational view of the package of FIG. 24 folded out to create two tilt stands, and showing a process of flushing air from the system using fluid that flows into a standard catch basin;

FIG. 26 is a plan view of an example delivery system package having a built-in immersion tank fitted around the delivery system shaft;

FIG. 27 is an enlargement of the immersion tank of the package of FIG. 26 filled with saline and showing a device being flushed or de-aired;

FIGS. 28A-28C illustrate an alternative immersion tank that can be provided as part of a delivery system package in three different use configurations; and

FIGS. 29A and 29B are schematic side views of an example delivery system package having a built-in immersion tank for bendable systems.

### DETAILED DESCRIPTION

The right ventricle and left ventricle are separated from the right atrium and left atrium, respectively, by the tricuspid valve and mitral valve; i.e., the atrioventricular valves. The septal wall extends between the right atrium and left atrium. The present specification and drawings provide aspects and features of the disclosure in the context of several implementations of replacement heart valves, delivery systems and methods that are configured for use in the vasculature of a subject (e.g., a living subject, a simulation, etc.), such as for treatment (such as replacement, repair, etc.) of natural heart valves in a subject. Valve treatment of the mitral or tricuspid valves is a primary focus of the examples in the present application, but characteristics of the delivery systems described herein may equally be used for other valve locations, and thus the claims should not be constrained to only mitral or tricuspid valve replacement unless expressly limited.

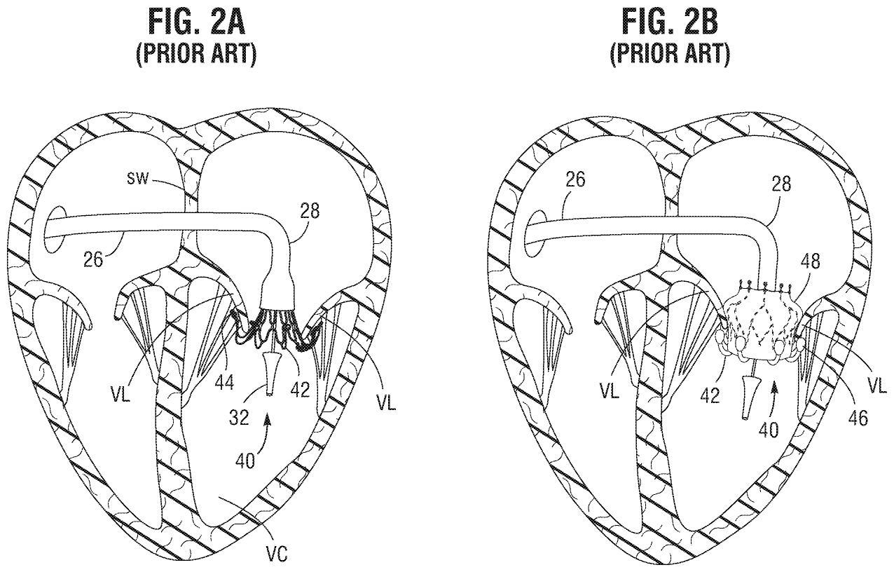

In some implementations, delivery systems described herein can be used for transfemoral percutaneous delivery of a treatment device, such as a replacement mitral valve or a repair device, to a mitral valve to treat patients with moderate to severe mitral regurgitation (or to a tricuspid valve to treat moderate to severe tricuspid regurgitation). In some cases, for safety and/or other reasons, the disclosed devices may be delivered from the atrial side of the atrioventricular valve or atrioventricular valve annulus. For example, a transatrial approach can be made through an atrial wall, which can be accessed, for example, by an incision through the chest. Atrial delivery can also be made intravascularly, such as from a pulmonary vein. The prosthetic valve can be delivered to the right atrium via the inferior or superior vena cava. In some cases, left atrial delivery can be made via a transeptal approach (e.g., FIGS. 2A and 2B). In a transeptal approach, an incision can be made in the atrial portion of the septal wall SW to allow access to the left atrium from the right atrium (the septum can be approached in one or more of a variety of transcatheter approaches, e.g., transjugularly, transfemorally, etc.). In short, the prosthetic valve can be delivered via transventricular, transatrial, transjugular, and/or transfemoral approaches with small or minimal modifications to the delivery process. Any of the approaches that travel through any vasculature can be call a transvascular approach as well.

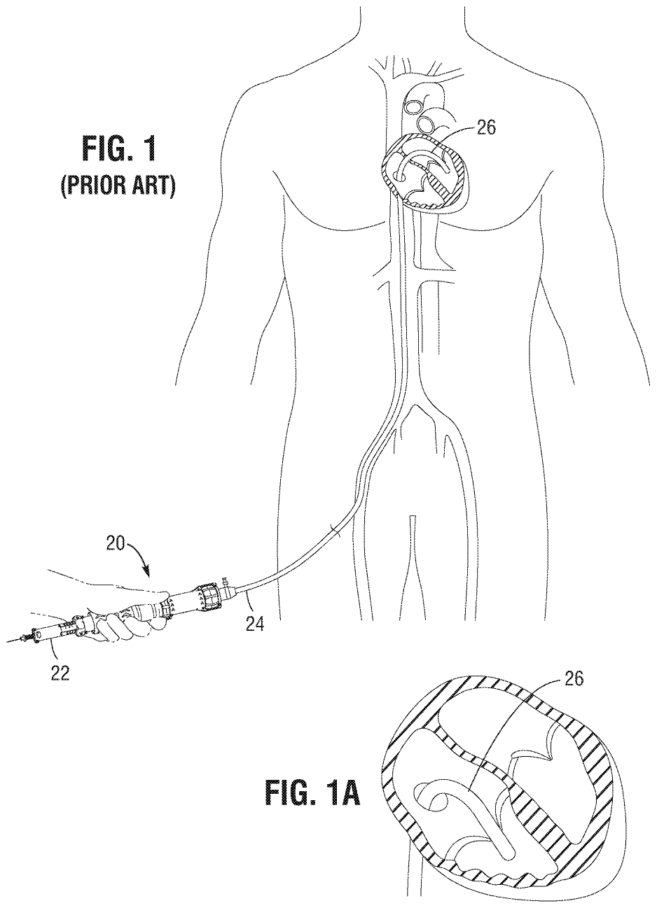

FIG. 1 illustrates a transcatheter mitral valve replacement procedure showing advancement of a delivery system 20 through a blood vessel in the leg such as the femoral vein up to the vicinity of the mitral valve within the heart. Example transfemoral approaches that can be used with the various implementations and inventive concepts herein can be found in U.S. Pat. Nos. 10,004,599 and 10,813,757, the entireties of which are hereby incorporated by reference for all purposes, but again, a variety of approaches are possible.

The illustrated prosthetic mitral valve delivery system 20 has a proximal handle 22 from which an elongated access sheath 24 extends distally. The access sheath 24 is shown extending into a lower portion of the venous system, such as into an ipsilateral femoral vein, and a catheter 26 advances from within the access sheath 24 up through the patient's venous system into the right atrium to access the tricuspid valve, or with a further transseptal puncture using known techniques into the left atrium to access the mitral valve. FIG. 1 shows the latter procedure where a distal segment 28 (see FIG. 2A) of the catheter 26 has crossed the septal wall SW. FIG. 1A shows a potential approach to access the tricuspid valve with the distal tip of a delivery system in the right atrium to access the tricuspid valve. Both procedures can be enhanced by the various system and assembly features, attributes, characteristics, preparations, methods, steps, etc. described herein, individually or in conjunction with one another. All combinations of features and steps as described herein are contemplated, assuming they are not mutually exclusive or redundant in deployment.

In some implementations herein, the access sheath 24 can be integrally associated with the proximal handle 22 or can be a separate instrument. In some implementations, an integral sheath 24 would be fixedly attached to the proximal handle 22, with the catheter 26 extending through and movable with respect to both the handle 22 and the sheath. With a separate sheath 24, in some implementations, the sheath can have a proximal hub with elastomeric valves and/or seals. In some implementations, the catheter 26 can be fixedly attached to the proximal handle 22 and passed through the valves or seals to prevent blood leakage. Both types of access sheath 24 are contemplated herein, and the claims should not be considered limited to one or the other unless specifically recited.

The proximal handles (e.g., handle 22, etc.) herein as well as attendant support systems such as guidewires and inflation connections can be configured in a variety of ways, and can comprise one, some, or all of the features seen in U.S. Pat. Nos. 10,004,599 and 10,813,757, incorporated by reference herein, in addition to any features described with respect to various implementations herein.

To better understand certain aspects of the improvements disclosed herein, an example mitral valve replacement procedure utilizing an example valve delivery system 20 will be described with reference to FIGS. 2A and 2B. The figures are labeled “PRIOR ART” relative to the basic steps illustrated. The same or similar steps or improved steps can be practiced with one or more of the inventive and new features/steps described herein with respect to the various implementations herein.

While some of the basic steps/features illustrated may be “PRIOR ART” in figures so labeled, some of the discussion and descriptions below regarding these and other figures herein may not be in the prior art, i.e., the descriptions and discussions below may include descriptions of steps and/or features and/or variations that are improvements over the prior art. In other words, just because a step or feature is described below in a discussion related to one or more figures labeled “PRIOR ART,” does not necessarily mean everything discussed is in the prior art.

To deliver the prosthetic valve to the native mitral valve annulus, the prosthetic valve can be radially crimped into a collapsed configuration within a catheter 26 of the delivery system 20. In some implementations, the prosthetic valve can fit inside of a 30 French (F) catheter (in a collapsed state). In some implementations, the prosthetic valve can be configured to fit into even smaller catheters, such as a 29 F, 28 F, 27 F, or 26 F catheter.

With reference to FIG. 1, the access sheath 24 of the delivery system 20 can be placed in the ipsilateral femoral vein and the catheter 26 advanced through the sheath toward the right atrium. A transseptal puncture through the septal wall SW using known techniques can then be performed to obtain access to the left atrium. The catheter 26 can then be advanced into the left atrium and then to the left ventricle. In some implementations, a guidewire can be used to position the catheter 26 in the proper position, and one or more guidewires can be used. In some implementations, a generally conical or otherwise tapered nose cone 32 can optionally be secured at the distal end 28 of the catheter 26 to help transit through the vasculature and other obstacles, such as the septal wall SW, as well as through the valve leaflets VL into the associated ventricular cavity VC.

It can be advantageous for a user to be able to steer the delivery system 20 through the complex areas of the heart in order to position a replacement mitral valve in line with the native mitral valve. For example, ability to steer or bend one or more portions of the sheaths/catheters may allow a user to manipulate the distal segment 28 of the catheter 26 to the appropriate area. The delivery system 20 can be advanced through the transseptal puncture and into the left atrium and can then further manipulate the delivery system 20 to create an even greater bend in the catheter 26. Further, a user can torque the entire delivery system 20 to further manipulate and control the position of the distal segment 28. The catheter 26 can be further advanced such that the catheter 26 (carrying the prosthetic valve) extends between the native leaflets of the mitral valve and into the left ventricle VC.

FIGS. 2A and 2B show an example prosthetic valve delivery, including expanding a prosthetic valve 40 using the catheter 26. In the delivery configuration, the catheter 26 is advanced or positioned over the collapsed prosthetic valve to convert or maintain the valve to a radially collapsed configuration. Although not shown, the prosthetic heart valve 40 can be mounted on the distal end of a delivery catheter having the capacity to displace the valve with respect to the catheter 26, or vice versa. In some implementations, a delivery system or delivery catheter can incorporate an expansion balloon, e.g., if the device is balloon-expandable.

FIG. 2B indicates partial retraction of the catheter 26 to expulse a plurality of ventricular anchors 42 in a circumferential array from a distal end of the prosthetic valve.

In some implementations, the heart valve 40 can be displaced relative to the catheter 26 such as by advancing a pusher device distally against the prosthetic valve and/or retracting the catheter sheath relative to the valve. In some implementations, terminal end portions 44 of the anchors 42 are biased to extend proximally (in the direction of a main body of the valve) when deployed. In some implementations, prior to rotation of the anchors 42, the constraining or restraining force applied by the catheter 26 on the anchors 42 can force the terminal end portions 44 to extend downward (away from the main body in a generally distal direction) during delivery.

In some implementations, once the prosthetic valve 40 is delivered to the native annulus region, the catheter 26 can be retracted farther relative to the prosthetic valve 40, thereby allowing the prosthetic valve 40 to expand radially outward. The release of the prosthetic valve 40 can be conducted in stages. In some implementations, the ventricular anchors 42 can be released from the catheter 26 (FIG. 2A) prior to the release of a main body portion of the valve 40, as seen in FIG. 2B. In some implementations, when the ventricular anchors 42 are released, they spread out away from the main body, with distal end portions 44 directed radially outward and upward. Subsequently, with release of the main body, the anchors 42 rotate toward the main body, such that the distal end portions 44 pivot toward the vertical (longitudinal) axis and wrap around behind the native leaflets VL on the ventricular side.

Optionally, the delivery system can be manipulated/adjusted to reposition the partially retracted valve 40 as desired, and retracts the catheter 26 further to cause the ventricular anchors 42 to engage the native valve annulus (FIG. 2A). Rounded head portions 46 of the ventricular anchors 42 can contact a ventricular side of the native valve annulus and/or adjacent tissue (such as trigone areas). In some implementations, the anchors 42 can be configured to point more directly upward upon full deployment, as compared to when they are partially deployed from the catheter 26. At this point, the user can assess engagement of the ventricular anchors 42 with the native valve annulus (such as through imaging means), prior to retracting the catheter 26 farther to deploy an atrial portion 48, as in FIG. 2B.

In some implementations, the catheter 26 can be retracted farther (not shown) to release the atrial portion 48 of the prosthetic valve 40, and the prosthetic leaflets begin functioning. In some implementations, the devices herein can be configured such that an atrial portion (e.g., such as atrial portion 48) forms a seal against the native annulus within the atrium. In some implementations, the device can be configured such that a seal created in the atrium by the atrial portion 48 and the seal created by the ventricular anchors 42 in the ventricle together prevent, reduce, or minimize the flow of blood between the native annulus and the outside of the main body during diastole and systole.

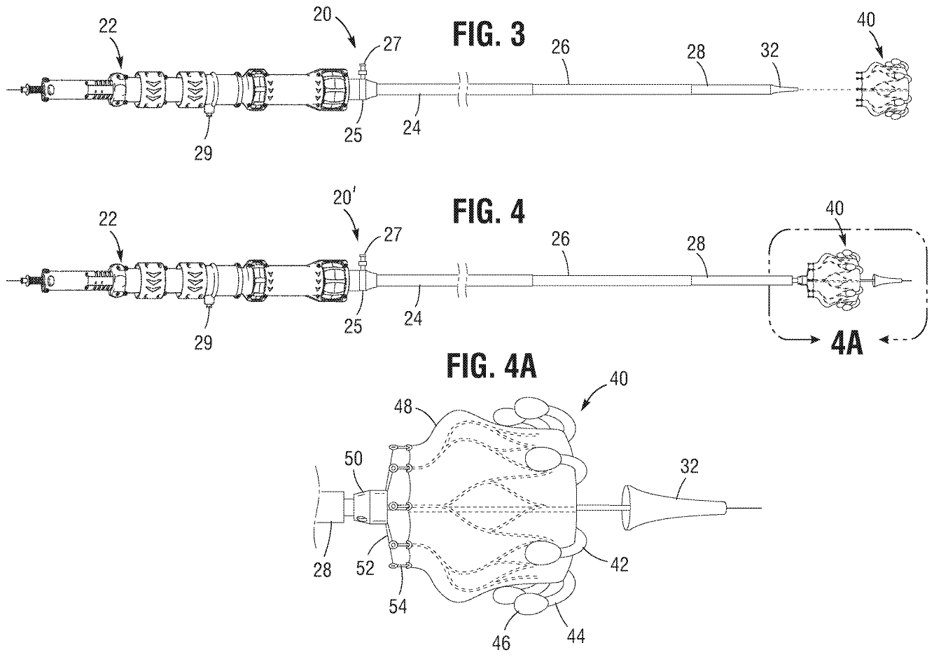

FIG. 3 is an elevational view of an implementation of an integrated transcatheter device (e.g., treatment device, implant, heart valve, prosthetic heart valve, repair device, etc.) and delivery system 20 (e.g., treatment device delivery system, implant delivery system, transcatheter delivery system, heart valve delivery system, repair device delivery system, etc.). In some implementations, the delivery system 20 has a proximal control handle 22 from which an elongated integrated introducer having an access sheath 24 around a concentric catheter 26 extends to a distal segment 28. In some implementations, the introducer with access sheath 24 is used to facilitate entry of the delivery system 20 into a vein for advancement through the vasculature to the heart.

In some implementations, the introducer has a proximal hub 25 in which is mounted a series of valves for sealing around the catheter 26. In some implementations, the proximal hub 25 has a fill port 27 for flushing internal passages therein. In some implementations, the delivery system 20 handle 22 also has a fill port 29 for flushing internal passages therein.

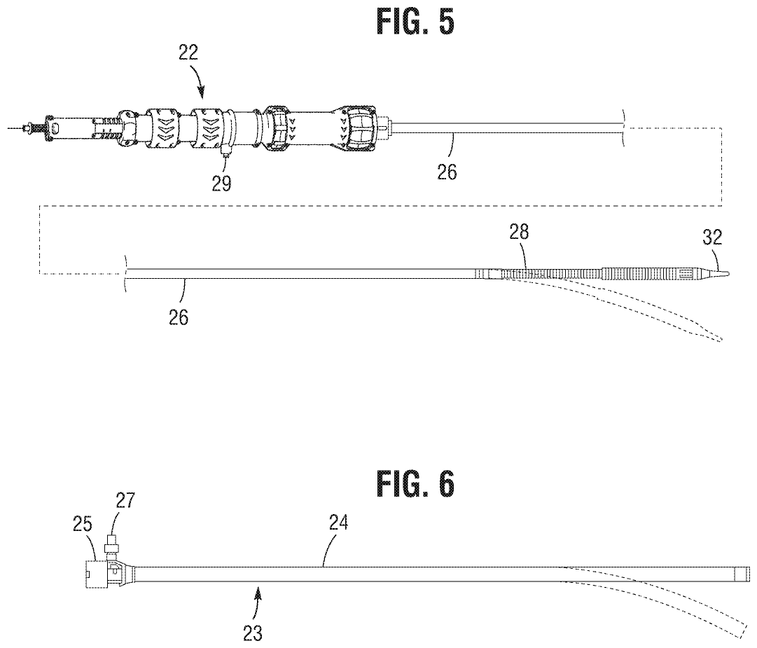

A system may be termed “integrated” when two or more separate components are provided together. In some implementations, an introducer having the access sheath 24 mounted onto the delivery system 20 is integrated, whereas FIGS. 5 and 6 described below show the parts separate. In some implementations, the distal segment 28 of the delivery system 20 forms a so-called capsule which contains the compressed device 40 (e.g., a prosthetic heart valve or other treatment device).

In some implementations, the capsule is too large in diameter to pass through a standard access sheath 24. In some implementations, the introducer is “integrated” with the delivery system 20 so that the capsule is located just distal of a distal end of the access sheath 24 and is passed into the vein at the same time as the access sheath. In some implementations, an optional tapered nose cone 32 facilitates this introduction. In some implementations, the integrated access sheath and delivery system 20 may be packaged together. In some implementations, the device 40 may be packaged separately, or the capsule may also have the device coupled to it, as in FIGS. 4 and 4A below.

In some implementations, the catheter 26 distal segment 28 is bendable, and controlled by one or more dials on the handle 22. In some implementation, the expandable device 40 is packaged separately and coupled to the integrated delivery system 20 just prior to the valve treatment procedure. In some implementations, the device 40 may have a self-expanding or a balloon-expanding support frame, the present application contemplating either. In some implementations, to couple the device 40 with the delivery system 20, the nose cone 32 is caused to advance from within the catheter 26, and the device either crimped around an inflation balloon or compressed and coupled with a sliding element (not shown) within the catheter. In some implementations, the control handle 22 features a number of dials or other actuators for moving the various concentric components axially relative to each other.

FIG. 4 is an elevational view of an implementation of an integrated transcatheter device and delivery system 20′ where the device 40 is tethered to a component of the delivery system, and 4B is an enlargement of the distal end of the delivery system showing the device depicted, for illustrative purposes, as a prosthetic heart valve (though other treatment devices can also be used). The delivery system 20′ shown may be identical to or similar to the implementation shown in FIG. 3, aside from the device 40 being supplied as an integrated component thereof.

With reference to FIG. 4A, the distal segment 28 of the delivery system catheter is shown with a retrieval hub 50 projecting therefrom. In some implementations, a plurality of flexible tethers 52, such as sutures, extend through the delivery system and emerge from a distal end of the retrieval hub 50. In some implementations, the tethers 52 each loop through an eyelet provided at a proximal end of each of a plurality of atrial stent struts 54 of the device 40. In some implementations, the tethers 52 loop through the eyelets and both free lengths are then threaded proximally back through a passage in the delivery system 20′ to the control handle 22.

Although not shown, in some implementations, the tethers 52 are secured to a movable element within the control handle 22 so the tension can be applied thereto. In this manner, the tethers 52 can be pulled in a proximal direction to cause the eyelets and stent struts 54 to be pulled into the retrieval hub 50. Thereafter, in some implementations, the entire device 40 can be pulled and compressed within the catheter distal segment 28, thus forming the capsule at the distal end of the access sheath 24.

FIG. 5 is an elevational view of a transcatheter delivery system 20″ by itself, without the introducer. As before, the delivery system 20″ has a proximal control handle 22 from which a catheter 26 extends terminating in the distal segment 28, and often having the capsule which contains the device 40. The distal segment 28 is commonly deflectable or steerable, as indicated in dashed line.

FIG. 6 is an elevational view of an introducer 23 separate from the delivery system of FIG. 5. In some implementations, the introducer 23 has the access sheath 24 extending distally from a proximal hub 25 in which is mounted a series of valves for sealing around the catheter 26 of the delivery system 20″. In some implementations, the introducer 23 may also have a distal segment that is deflectable or steerable, as indicated in dashed line. In this separate arrangement, the introducer 23 may first be passed into the vein by surgical cut down or percutaneously using the Seldinger technique, typically using a tapered tip mandrel or other such lead-in device. Subsequently, the delivery system 20″ is inserted through the proximal hub 25 and through the access sheath 24 before it emerges from the distal end of the sheath in the body. This sequence occurs when the capsule holding the device or heart valve at the end of the delivery system 20″ is small enough to pass through the introducer 23. In some implementations, the delivery system 20″ and introducer 23 would have first been independently flushed with fluid using the fill ports 27, 29 to remove air bubbles, as described elsewhere herein.

In some implementations, the delivery system 20″ is first inserted into the introducer 23 and then the assembly flushed with fluid using the fill ports 27, 29 to remove air bubbles, as described elsewhere herein. Then the assembly may be inserted into the vein.

Still further, when the capsule holding the device at the end of the delivery system 20″ is too large to pass through the introducer 23, a delivery system 20″ which is not packaged with the device (e.g., treatment device, heart valve, etc.), such as the integrated system of FIG. 3, is first inserted into the introducer 23. Then the device 40 is crimped down into the capsule which projects from the distal end of the access sheath 24. Subsequently, this assembly is flushed with fluid.

In some implementations, the delivery system 20″ (or any described herein) may be flushed and then inserted into the vein without using an introducer 23. In that case, the delivery system 20″ is the only assembly that requires flushing.

To summarize, the delivery system 20″, introducer 23 and device 40 may all be packaged in one integrated assembly and flushed together using the fill ports 27, 29. Or, the delivery system 20″ and introducer 23 are packaged together in an integrated unit, then coupled with a separate device 40 and the assembly flushed. Still further, the delivery system 20″ and device 40 may be packaged together, separate from the introducer 23, then assembled with the introducer 23 and flushed all together.

In some implementations, the delivery system 20″ and device 40 may be packaged together, flushed, then passed though the introducer 23 that has been separately flushed. Further, the delivery system 20″, introducer 23 and device 40 may all be packaged separately, then assembled and flushed together, or the delivery system 20″ and device 40 coupled and flushed separate from the introducer 23.

In some implementations, if no introducer is used, then the delivery system 20″ and device 40 may be packaged separately or together, then flushed as a unit. In short, there are a variety of ways to package and flush the three components, all of which could utilize the flushing techniques disclosed herein.

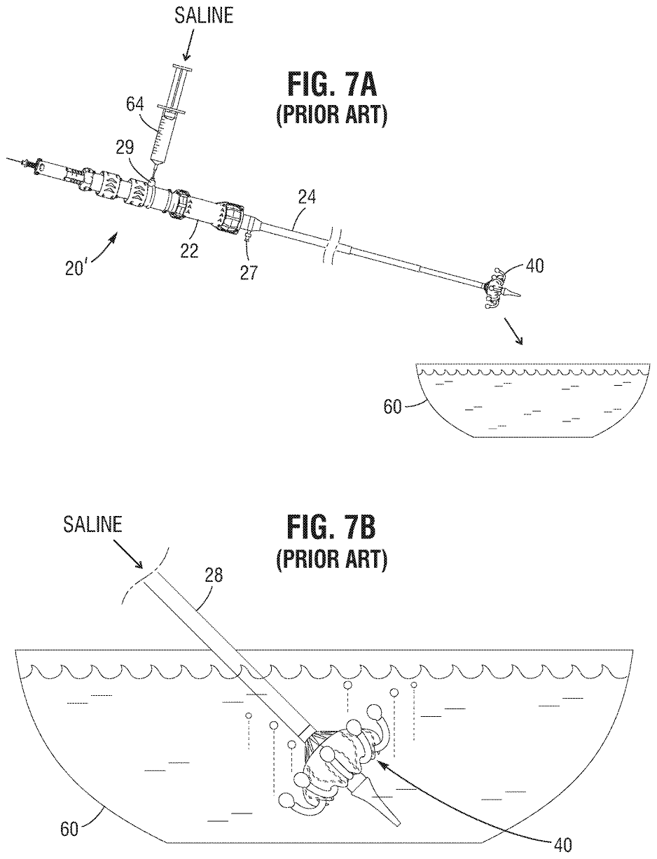

FIGS. 7A and 7B illustrate two steps in a process used to de-air or flush a transcatheter delivery system 20′ of FIG. 4. The delivery system catheter 26 is angled distal end downward into an immersion tank or bowl 60 filled with saline. The device 40 (e.g., a treatment device, a replacement device, a repair device, an implant, a prosthetic heart valve, etc.) may be partially extended from the distal segment 28 of the catheter, or may be fully extended in the case of a tethered valve. The proximal handle 22 of the delivery system has a flush port 29 to which a source of saline such as a syringe 64 may be coupled in a sterile manner.

FIG. 7B shows the device 40 immersed in the tank 60 while saline is injected into the flush port 29. The saline can flow downward through the delivery system pushing air with it, until it reaches the device 40 at which point air is expelled from the catheter distal segment 28.

In some implementations, by manipulating the device 40 in and out of the distal segment 28, a proper flush can be obtained of the fabric and other components of the device to ensure that any trapped bubbles are released, as shown.

The entire process of flushing air from the system and device may be manual and time-consuming even though delay can be a real issue. In some implementations, the process takes place in the sterile environment of an operating room after the delivery system and device, together or separately, are removed from their packaging and coupled together. Additional supplies such as the immersion bowl 60, saline, syringes, towels must be provided by the operating room hospital, making the surgical team seem unprepared or burdensome to hospital staff. Further, multiple steps and holding the relatively lengthy delivery system 20′ in this manner while injecting saline into the flush port 29 is extremely awkward, sometimes requiring two people. Improper manipulation of the delivery system by a user could potentially lead to a breach of the sterile field. Improper de-airing of the system and improper device loading may lead to other issues, including damage to the system. Inadvertent spills could be messy, and disposable supplies that may be involved could contribute to waste. Consequently, there is a need for a more expedited and efficient flushing systems and processes.

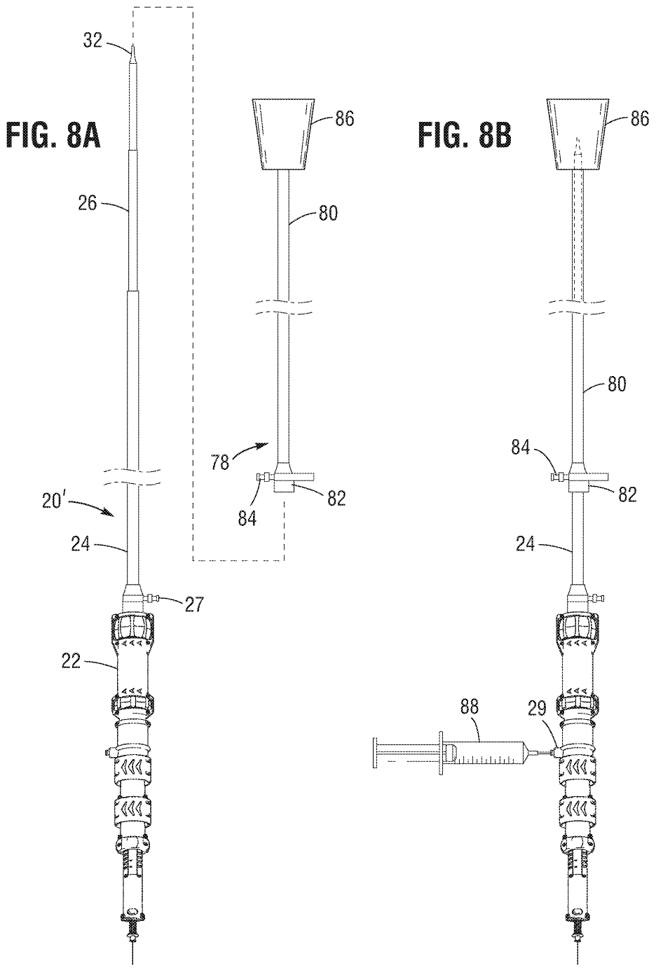

FIG. 8A is an exploded view showing a delivery system 20′ (e.g., transcatheter delivery system, treatment device delivery system, transcatheter heart valve delivery system, repair device delivery system, device delivery system, implant delivery system, etc.) next to a de-airing or flushing implement 78. In some implementations, the flushing implement 78 comprises an elongated tube 80 terminating at a lower or proximal end in a hub 82 having a fluid port 84. In some implementations, an immersion tank 86 is provided at an upper or distal end of tube 80.

FIG. 8B shows an assembled view of the two components, with the delivery system catheter 26 being inserted upward into the hub 82 and through the tube 80 until the nose cone 32 resides within the immersion tank 86. Again, a saline flushing syringe 88 is then coupled to the flush port 29 on the delivery system control handle 22.

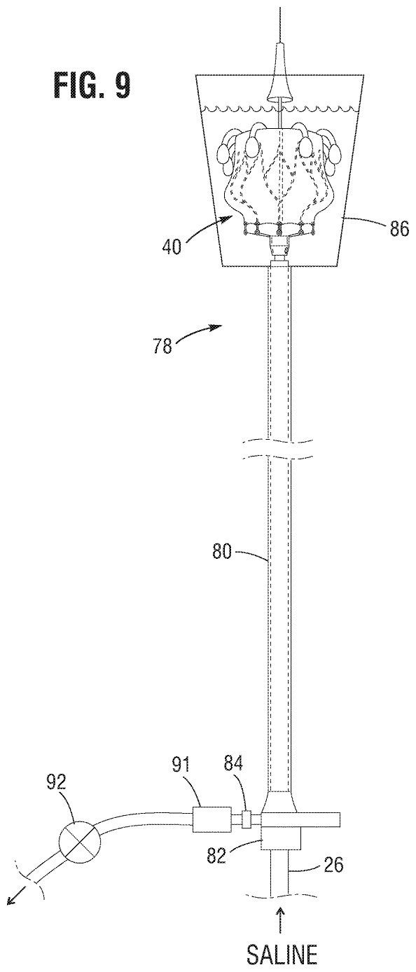

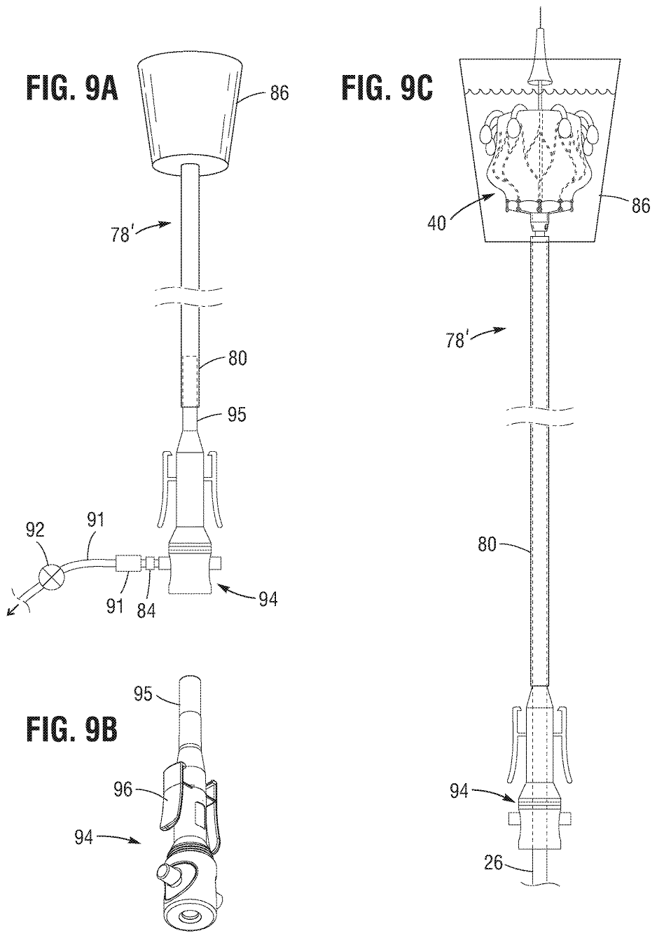

FIG. 9 is an enlargement of the flushing implement 78 assembled with the delivery system 20′ with the device 40 (e.g., treatment device, implant, replacement device, heart valve, prosthetic heart valve, repair device, etc.) expanded within the immersion tank 86. Saline has been introduced using the syringe 88 through the flush port 29 and injected upward to fill the immersion tank 86 to a level above that of the expanded device 40.

In some implementations, a fluid coupling and line 91 are attached to the fluid port 84. In some implementations, the fluid coupling and line 91 have a fluid release valve 92 therein which permits controlled release of saline from the flushing implement 78 to prevent overflow at the top of the immersion tank 86. That is, saline flush pushed through the delivery system catheter 26 enters the immersion tank 86, and then can flow by gravity through the concentric space between the catheter and the tube 80, which opens to the fluid port 84.

FIG. 9A is an exploded view of an optional flushing implement 78′, and FIG. 9C is the flushing implement assembled with the delivery system and showing a device 40 (depicted as a heart valve, but other treatment devices are also possible) expanded within an immersion tank 86. While the first flushing implement 78 was shown with an integrated hub 82, tube 80, and immersion tank 86, the alternative implement 78′ has just the tube 80, and no hub, but a separate loader 94 used to facilitate introduction of the delivery system. As seen in FIG. 9B, the loader 94 has a tapered forward nose piece 95 adapted to fit tightly into the tube 80, as seen, which prevents leakage.

In some implementations, insertion of the delivery system catheter 26 into the tube 80 is facilitated by first mounting the loader 94 onto a distal end of the delivery system and then using the nose piece 95 to connect to the tube 80 before advancing the delivery system. In some implementations, the loader 94 is also provided with a fluid line 91 leading to a fluid release valve 92, as before. In some implementations, the loader 94 has a pair of cantilevered fingers 96 arranged to clasp onto a proximal hub of an access sheath when later used to facilitate introduction into the body.

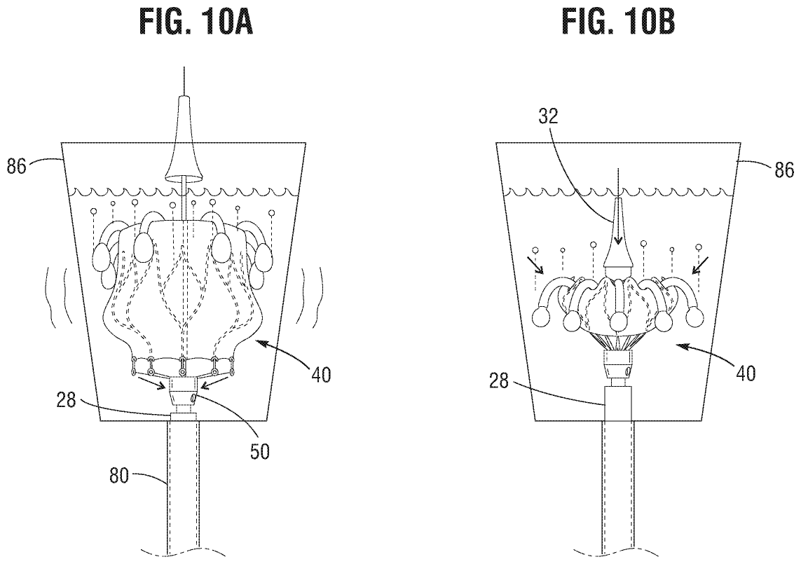

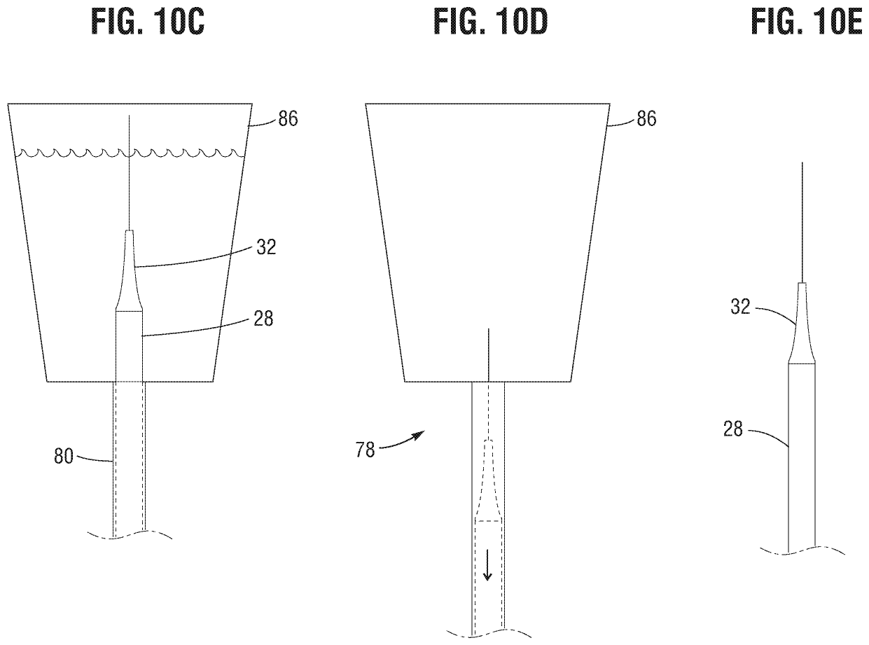

FIGS. 10A-10E are enlargements showing steps in flushing or de-airing of the delivery system 20′ and device 40 within the immersion tank 86 and removal of the delivery system therefrom. FIG. 10A shows agitation of the immersion tank 86 to encourage release of air in the form of bubbles from within the delivery system and that may be trapped in the features of the device 40. The device 40 can be partially or fully expanded as shown within the immersion tank 86, and eventually contraction and retraction into the delivery system is commenced. FIG. 10B shows partial collapse of the device 40 into the retraction hub 50. Ultimately, the entire device 40 is retracted into the delivery system, and the nose cone 32 pulled back to form a cap of sorts on the delivery system. The nose cone 32 is typically formed of a soft material and facilitates advancement of the delivery system catheter 26 through the vasculature and into the heart.

FIG. 10C shows full retraction of the device 40 into the distal segment 28 of the elongated tube 80. At this stage, the delivery system is retracted from the flushing implement 78, as seen in FIG. 10D. This may be preceded by emptying the immersion tank 86 through the fluid release valve 92 (see FIG. 9). FIG. 10E shows the distal segment 28 of the delivery system fully removed from the flushing implement 70 and ready for use in delivering the device to the target annulus.

The vertical flushing solution disclosed above greatly simplifies the process of flushing or de-airing the delivery system and device (e.g., prosthetic heart valve, treatment device, repair device, etc.). However, the present application also contemplates a similar system integrated with the packaging used to ship the delivery system by itself, or with the packaging for the delivery system pre-coupled to the device (e.g., prosthetic heart valve or other treatment device). The various concepts disclosed herein for packaging the flushing system components with the delivery system facilitates the flushing operation by speeding up the process and eliminating the need for additional supplies from the hospital, except perhaps for saline.

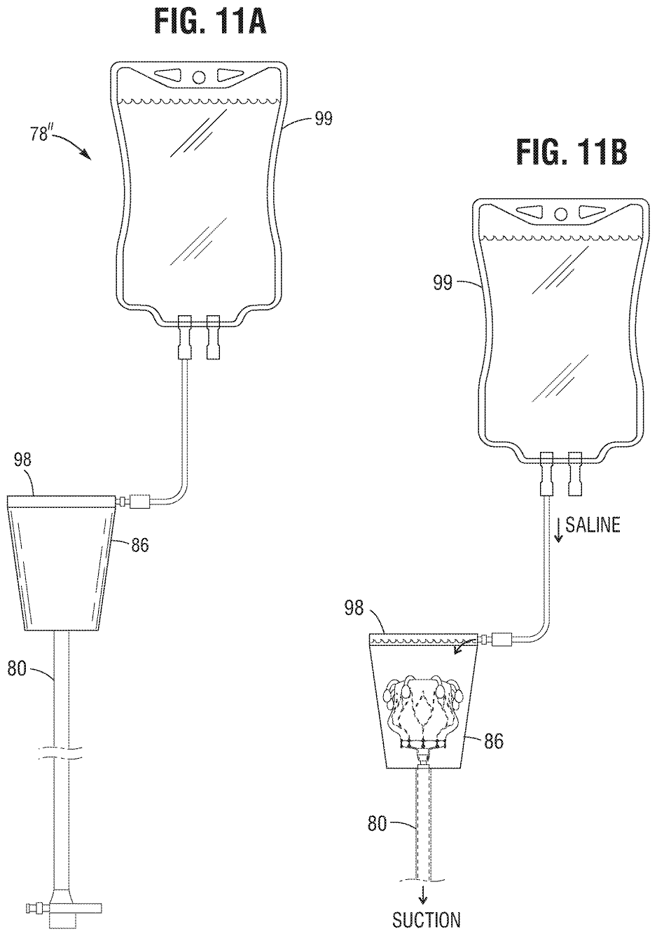

FIG. 11A is a schematic view of an alternative flushing assembly 78″ wherein saline fills an immersion tank 86 through suction and gravity. The immersion tank 86 has an upper cover 98 to which a supply of saline 99 attaches. In some implementations, the supply of saline 99 may flow in through a port near the top of the immersion tank 86. FIG. 11B illustrates the alternative flushing assembly 78″ in use with saline flowing through the cover 98 and into the immersion tank 86. The saline is encouraged to fill the immersion tank 86 from the supply of saline 99, such as a fluid bag held above the assembly 78″, by pulling suction through the elongated tube 80. The suction can be drawn through the delivery system by attaching a syringe to the previously-described flush port 29 on the control handle (see, e.g., FIG. 8B).

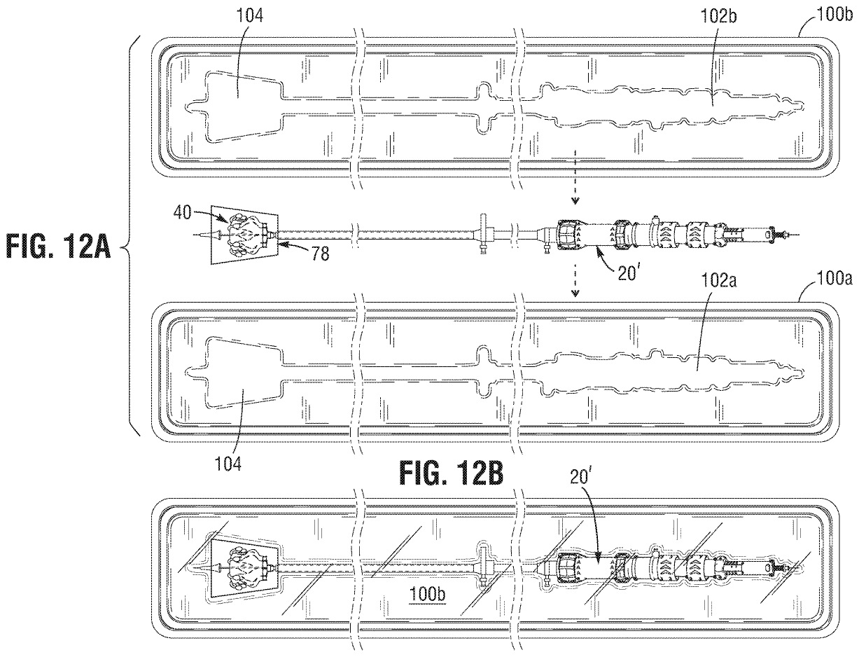

FIG. 12A is an exploded plan view of a delivery system 20′ assembled with the flushing implement 78 and packaging therefor. More particularly, the packaging comprises bookend lower and upper plastic trays 100a, 100b that are molded to accommodate the shapes of the delivery system 20′ assembled with the flushing implement 78. More particularly, the lower tray 100a has an elongated recess 102a molded therein which conforms to the exterior shape of the assembled delivery system 20′ and flushing implement 78. Likewise, the upper tray 100b has a similar but mirror image recess 102b. One or both of these recesses 102a, 102b defines an enlarged cavity 104 shaped to conform to the immersion tank 86. The lower recess 102a may be deeper than the upper recess 102b, or the shapes may be the same. The assembly of the delivery system 20′ and flushing implement 78 are placed into the lower recess 102a, and the upper tray 100b is then placed thereover and sealed around the outer edges, to result in the fully packaged view of FIG. 12B.

It should be noted that the particular delivery system 20′ shown has the device 40 integrated therewith, but a similar packaging solution can be used for the delivery system 20 of FIG. 3 without the device 40, which can be shipped separately. In that instance, prior to flushing/de-airing the system, the device 40 is coupled to the distal segment of the delivery system 20′.

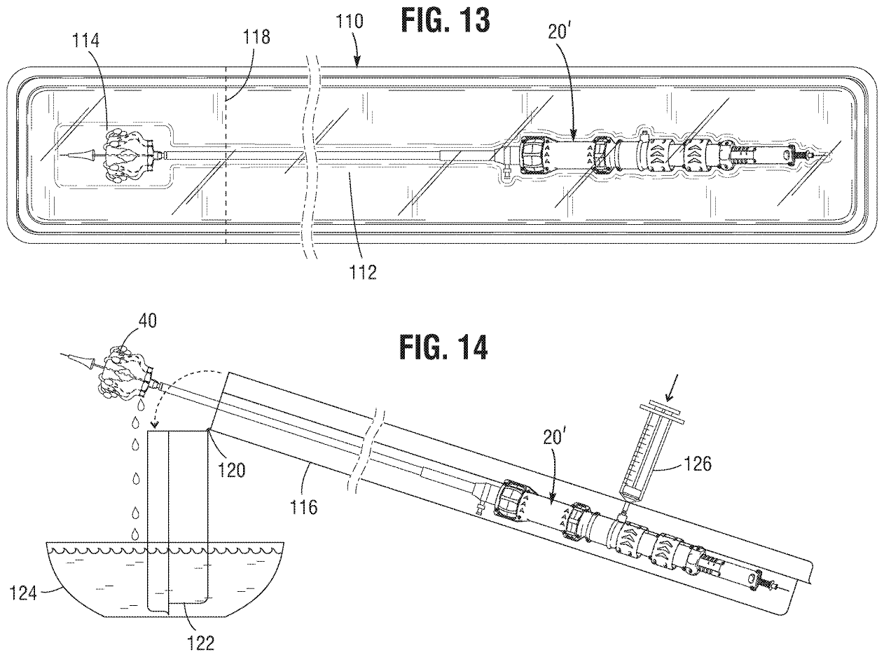

FIG. 13 is a plan view of a delivery system 20′ in a modified package 110. One or both of the two halves of the package 110 have a molded recess 112 shaped to closely receive the system 20′ including a distal chamber 114 large enough to accommodate the device 40 in its expanded configuration. In some implementations, if a paper or plastic card is used for packaging rather than a thermoformed plastic tray, no molded recess is required for the device. In some implementations, there is no separate flushing implement 78 such as shown in FIG. 12A.

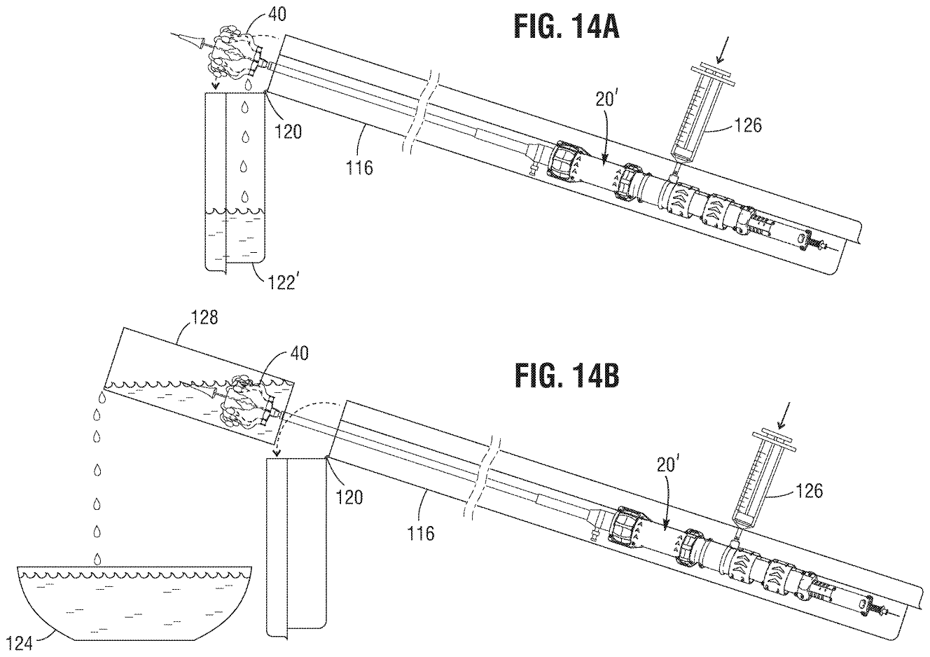

In some implementations, a lower half 116 of the package 110 is cut or otherwise scored along a lateral line 118 so as to form a living hinge 120 that enables folding down of a distal portion 122 of the lower half, as seen in FIG. 14. In some implementations, once the upper half of the package is removed, a distal portion 122 of the lower half 116 is folded down to provide a tilt stand for the entire package 110 so that the device 40 at the distal segment of the delivery system 20′ can be elevated for flushing; preferably over a catch basin 124 as shown. A majority of the delivery system 20′ remains held by the rest of the package 110, which frees up the hands of the user to inject the saline through the syringe 126. Once the system 20′ and device 40 have been flushed, and the device retracted into the delivery system, the system is then removed from the lower half 116 of the packaging which can be disposed of with minimal waste.

It should be noted that the various folded sections of packaging disclosed herein can be formed by a number of hinge means. For instance, plastic tray packaging can be scored or partially cut to define a living hinge, or the plastic in that lateral plane might be simply an unreinforced thin-walled section more prone to bend than thicker adjacent parts. Card-type trays may also be used, with folds in the card being similarly formed.

FIGS. 14A and 14B are variations of the delivery system and modified package of FIG. 13. In FIG. 14A, the distal portion 122′ of the lower half of the package 110 acts as a catch basin, obviating the need for a separate basin. The device 40 at the distal segment of the delivery system 20′ does not project as far out of the rest of the package 110 so that fluid drips into the integrated catch basin formed by the distal portion 122′.

FIG. 14B shows an alternative where an immersion tank 128 is supplied in the package 110 around device 40. The package 110 is once again scored or cut to enable folding down of the distal portion 122 of the lower half to form the tilt stand, and the delivery system 20′ and device 40 can be flushed with the device being immersed in fluid, as in the implementation shown in FIGS. 10A-10E. A separate catch basin 124 may be needed to prevent a mess.

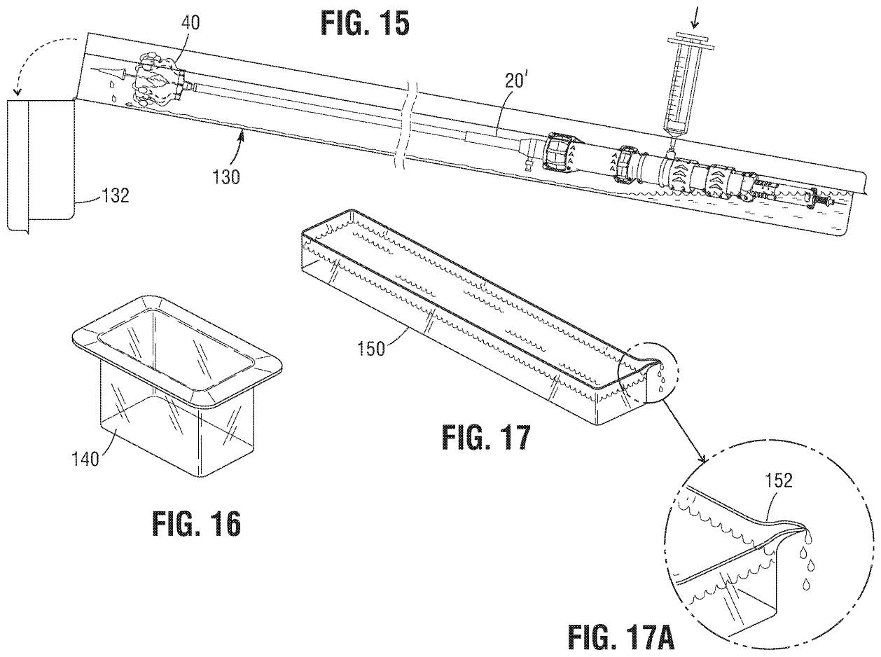

FIG. 15 is a side view of a delivery system 20′ and further modified package 130 with a distal portion 132 folded to provide a tilt stand therefore. In contrast to FIG. 14, the delivery system 20′ and device can be flushed/de-aired while still within the package 130. That is, the length of the package 130 is greater than the length of the delivery system 20′ such that the flushing operation can be accomplished without removing the system from the package, and without dripping saline into the surrounding environment. Once flushed, the system 20′ is removed from the package 130 for use, and the saline is poured down the drain.

FIG. 16 is a perspective view of an example drip tray 140 sized to fit within a delivery system package. The tray 140 may be sized and shaped to fit around or adjacent to the delivery system 20′. This obviates the need for a separate drip tray supplied by the operating room hospital.

FIG. 17 shows an example delivery system package 150 modified with a pour spout 152 on one corner, and FIG. 17A is an enlargement of the pour spout. With a package such as shown in FIG. 15, where the flushing occurs within the package, the saline must be poured off, and the package is somewhat bulky, elongated and flexible. Providing the pour spout 152 facilitates this disposal.

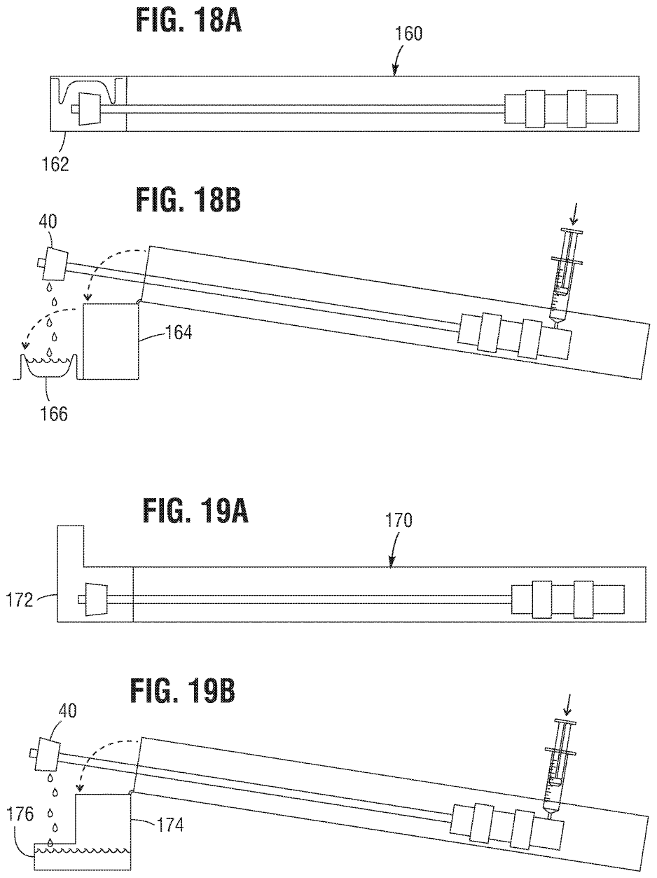

FIG. 18A schematically illustrates a delivery system package 160 with hinged components for creating a self-contained tilt stand. FIG. 18B is a side view showing a distal end portion 162 folded down about a living hinge to create a tilt stand 164. A connected portion that is initially folded up over the device 40 creates a built-in drip tray 166 when folded 90° from the tilt stand 164. The positioning of the drip tray 166 directly under the device 40 enables flushing of the system without any mess. Again, the entire package 160 can then be simply disposed of once the saline is poured out.

FIG. 19A is a side view schematically illustrating a delivery system package 170 also having hinged components. FIG. 19B shows a side view after a single hinged portion 172 is folded outward such as about a living hinge to create both a tilt stand 174 and a built-in drip tray 176. Again, the drip tray is positioned directly under the device 40 to catch the flush fluid, but may incorporate the volume within the tilt stand 174 for added capacity.

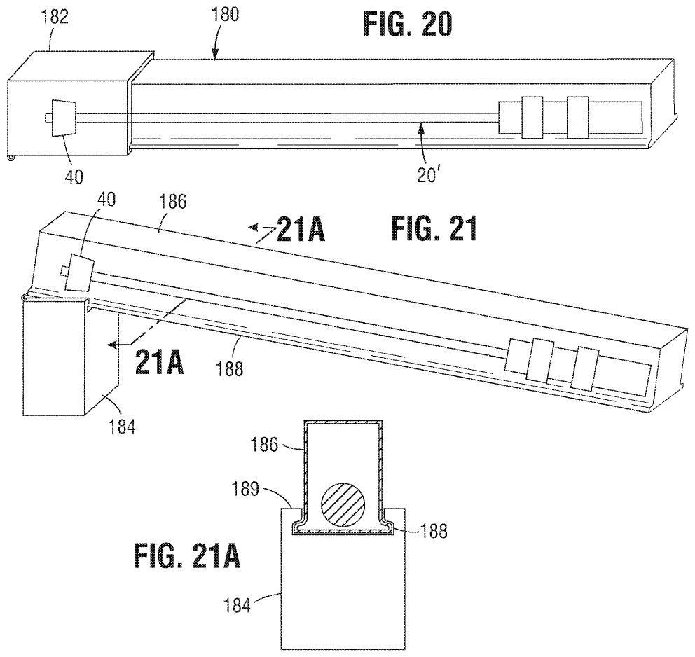

FIG. 20 schematically shows a delivery system package 180 having a hinged tilt stand incorporated therein. In particular, a distal portion 182 connects with the main part of the package 180 about a living hinge and can be folded 270° around the end of the package as seen in FIG. 21 to form a tilt stand 184. The main part of the package 180 and the distal portion 182 cooperate to secure the tilt stand 184 in place.

In some implementations, the main part of the package may have outwardly-projecting rails 188 which snap into a pair of flanking inwardly-projecting rails 189 on the distal portion 182 in an interference fit, as seen in FIG. 21A. This secures the tilt stand 184 relative to the rest of the package 180 for stability. It should be understood that this configuration or other such mating structure can be incorporated into any of the folding tilt stands and other folding elements of the packages described herein.

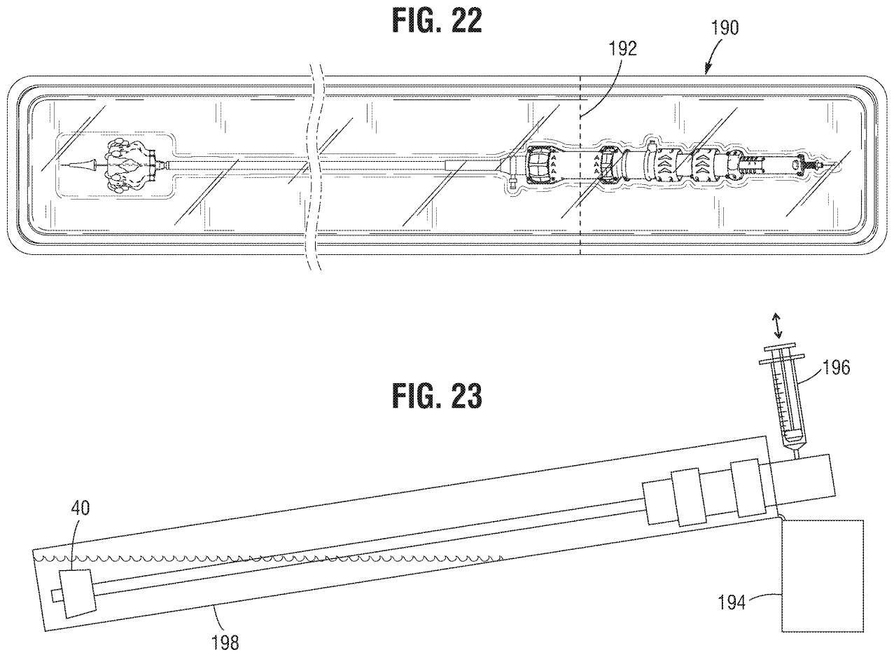

FIG. 22 is a plan view of a delivery system package 190 having a lateral score line 192 at the location of the control handle of the delivery system. FIG. 23 shows a proximal portion of the package folded down to create a tilt stand 194. In this configuration, the distal end of the package 190 is at the lower end and saline can be introduced to immerse the device 40. The distal end of the package forms a reservoir 198 to facilitate aspiration of fluid and bubbles from the device and through the delivery system, such as by withdrawing the plunger on a syringe 196 connected to a flush port in the handle. Thus, the flushing/de-airing operation can be done without removing the system from the packaging at all. In some implementations, fluid may be introduced into the control handle using the syringe 196 to flush the system and device 40, with the fluid being captured within the distal reservoir 198.

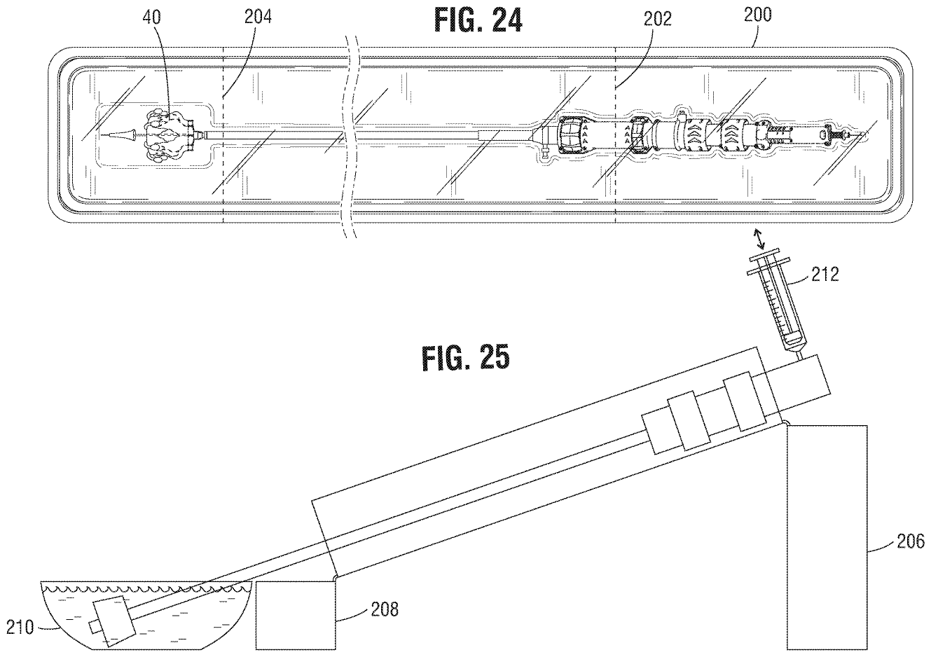

FIG. 24 is a plan view of a delivery system package 200, and FIG. 25 is a side elevational view thereof after having been folded out. The package 200 incorporates a pair of lateral score lines 202, 204; one that is located across the control handle of the delivery system, and one just proximal to the device 40. The score lines 202, 204 enable tilt stands 206, 208 to be formed when folded about living hinges. The proximal tilt stand 206 is taller than the distal tilt stand 208, so that the package and delivery system therein is tilted with the device 40 downward. By positioning the device 40 in an immersion tank 210, saline can be introduced or withdrawn from the system without mess and without requiring holding, such as by using the syringe 212.

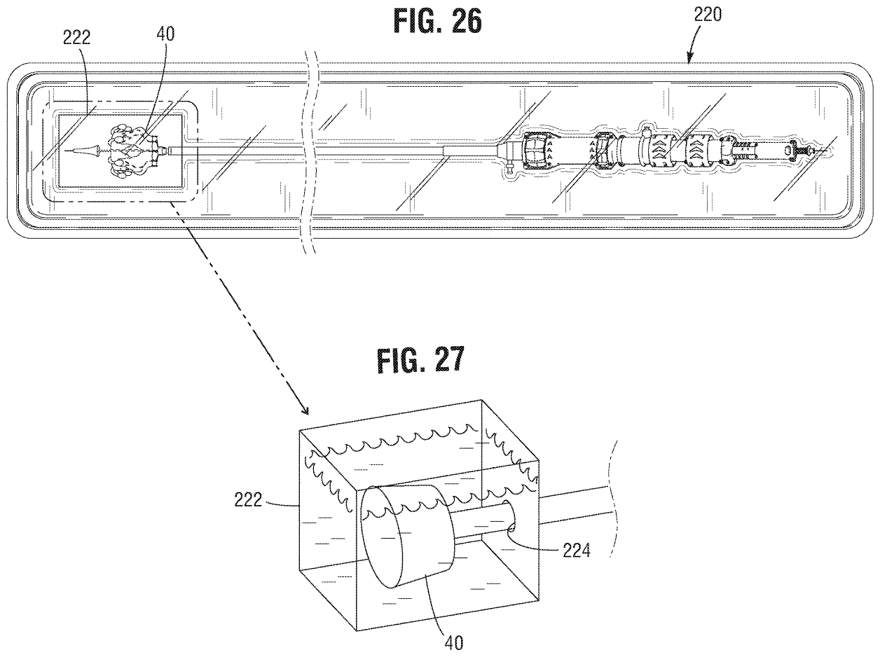

FIG. 26 is a plan view of a delivery system package 220 having a built-in immersion tank 222 fitted around the delivery system shaft. That is, one or both halves of the package 220 define an enlarged recess that accommodates the immersion tank 222. The delivery system and immersion tank 222 can be removed from the package, wherein FIG. 27 shows the immersion tank 222 filled with saline. The delivery system passes through a sealed aperture 224 to prevent leakage from within the immersion tank 222. In this arrangement, the device 40 can be de-aired while immersed in the tank 222. Once the flushing is complete, the device 40 is constricted and retracted within the delivery system sheath, which can then be removed through the sealed aperture 224 from within the tank 222.

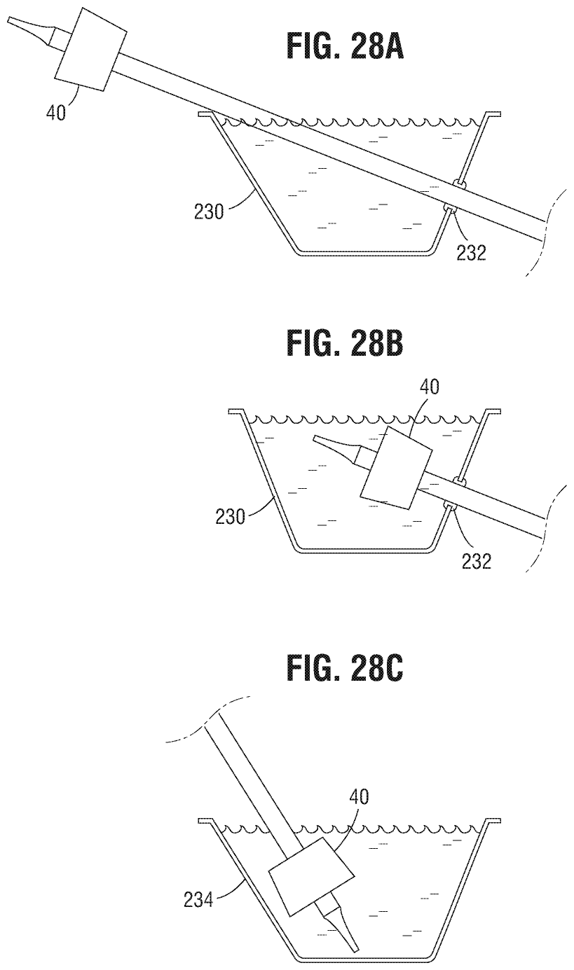

FIGS. 28A-28C illustrate implementations of immersion tanks that can be provided as part of a delivery system package in multiple different use configurations. FIGS. 28A and 28B show a first immersion tank 230 with a sealed aperture 232 through one of a number of angled walls. Using angled walls reduces the amount of fluid needed for immersion, and also enables the delivery system to be inserted through the aperture 232 and the device 40 raised above the immersion tank 230 for inspection. FIG. 28B shows a version where the device 40 is immersed in saline within the tank. FIG. 28C shows an immersion tank 234 with angled walls but no aperture in the sidewalls. This just provides a simple immersion tank or bowl to use for the process of flushing or de-airing the delivery system and device.

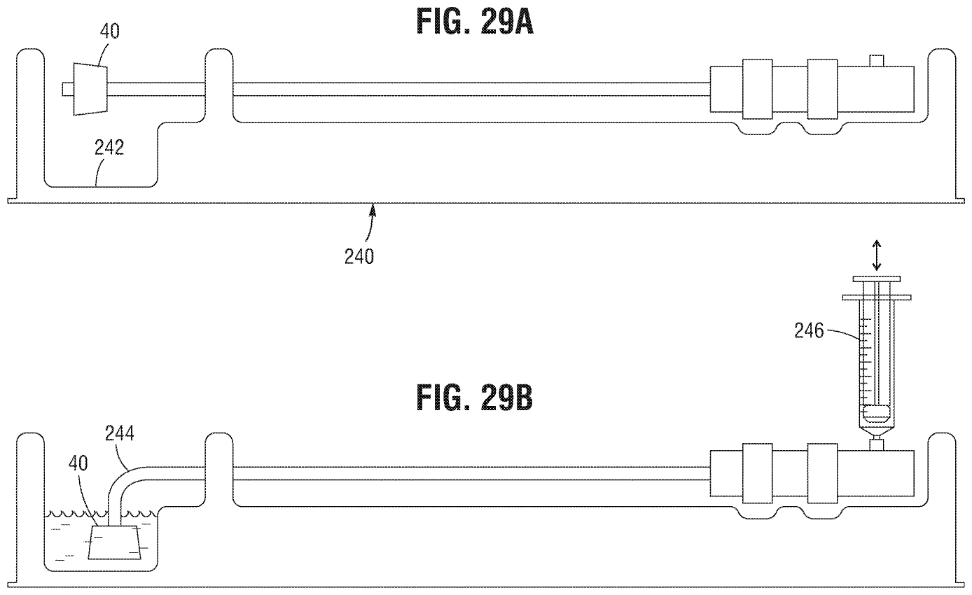

Finally, FIGS. 29A and 29B are schematic side views of a delivery system package 240 having a built-in immersion tank for bendable systems. Namely, the device 40 is held above a deep recess 242 having sidewalls (not shown). The device 40 can be immersed in saline within the recess 242 by creating a bend 244 in the delivery system access sheath as shown. This enables saline to be injected or aspirated using a syringe 246 without removing the system from the packaging at all.

In some implementations, the packages or packaging herein can be plastic thermoformed packages. In some implementations, the packages or packaging herein can have a flexible foldable portion on one or both ends to create the elevation needed for tilting and/or elevating to the top of a saline bowl. In some implementations, the packages or packaging herein can have tall side walls elsewhere to give them bending resistance over the sections of elevated span, so they don't droop or sag.

In some implementations, the packages or packaging herein includes a paper or plastic card with various fold-up flaps and straps to secure the device to the card during shipping, rather than a thermoformed plastic tray. The tilting concepts described herein could also be adapted for a card, though some additional features may be included. In some implementations, card packages can have a flexible foldable portion on one or both ends, as well as having tall side walls elsewhere to give them bending resistance. The flexible hinge in some places with rigid portions elsewhere could be integrated into a card design through, for example, the use of triangular fold-up side supports and a retractable support tube.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

The techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

While the foregoing is a complete description of preferred implementations of the invention, various alternatives, modifications, and equivalents may be used. Moreover, it will be obvious that certain other modifications may be practiced within the scope of the appended claims.

What is claimed is:

1. An assembly, for use with a fluid, the assembly comprising:
an implant;
a delivery system including:
a catheter defining an inner lumen therethrough;
a control handle that extends from a proximal end of the catheter, and that has a fluid port in communication with the inner lumen; and
a distal part, coupled to the implant; and
packaging:
including a first portion and a second portion;
containing the implant and the delivery system such that the implant is housed in the first portion and the handle is housed in the second portion; and
having a lateral line that connects the first portion to the second portion in a manner that enables the first portion to be rotated relative to the second portion to form the packaging into a tilt stand in which, within the packaging, the delivery system is held in a tilted repose to facilitate flushing of the delivery system and the implant with the fluid via the fluid port.

2. The assembly according to claim 1, wherein the tilt stand tilts the delivery system in a manner that elevates the implant above the control handle.

3. The assembly according to claim 1, wherein the tilt stand tilts the delivery system in a manner that elevates the control handle above the implant.

4. The assembly according to claim 1, wherein the second portion includes an inwardly projecting rail and the first portion includes an outwardly projecting rail that mates with the inwardly projecting rail to form the tilt stand.

5. The assembly according to claim 1, wherein rotating the first portion to form the tilt stand exposes the implant while the control handle remains contained within the second portion.

6. The assembly according to claim 1, wherein the lateral line includes a hinge about which the first portion is rotatable relative to the second portion.

7. The assembly according to claim 1, wherein the fluid is injectable into the fluid port from outside the packaging while the delivery system remains within the packaging.

8. The assembly according to claim 1, wherein, in the tilted repose, the fluid flows upward from the fluid port through the handle.

9. The assembly according to claim 8, wherein, in the tilted repose, the fluid pools in the first portion.

10. The assembly according to claim 8, wherein, in the tilted repose, the fluid pools in the second portion.

11. The assembly according to claim 10, wherein the second portion includes a spout for spilling of pooled fluid.

12. The assembly according to claim 1, wherein, in the tilted repose, the fluid flows downward from the fluid port through the handle.

13. The assembly according to claim 12, wherein the tilt stand is configured such that the fluid pools in the first portion.

14. The assembly according to claim 13, wherein, in the tilted repose, the tilt stand tilts the assembly such that the implant becomes submerged in the pooled fluid.

15. The assembly according to claim 1, wherein the lateral line is a first lateral line and the second portion includes a second lateral line that splits the second portion into a third portion and a fourth portion, the second lateral line connecting the third portion to the fourth portion in a manner that enables the fourth portion to be rotated relative to the third portion.

16. The assembly according to claim 15, wherein, in the tilted repose, the tilt stand tilts the assembly in a manner that elevates the control handle above the implant and such that the implant is extendable through the packaging into a drip tray.

17. The assembly according to claim 1, wherein the packaging further includes a drip tray enclosed within the first portion.

18. The assembly according to claim 17, wherein the drip tray is integral with the first portion.

19. The assembly according to claim 1, wherein the first and second portions include respective upper and lower trays that are molded to accommodate shapes of the delivery system and implant, and that are sealed to each other to form the packaging.

20. An assembly, comprising:
an implant;
a delivery system including:
a catheter defining an inner lumen therethrough;
a control handle that extends from a proximal end of the catheter, and that has a fluid port in communication with the inner lumen; and
a distal part, coupled to the implant; and
packaging:
including a first portion, and a second portion;
containing the implant and the delivery system such that the implant is housed in the first portion and the handle is housed in the second portion; and
having a hinge that hingedly connects the first portion to the second portion to enable the first portion to be rotated relative to the second portion, to form the packaging into a tilt stand in which, within the packaging, the delivery system is tilted to facilitate flushing of the delivery system and the implant via the fluid port.
